(19) United States
(12) Patent Application Publication
(10) Pub. No.: US 2024/0078791 A1
(43) Pub. Date: Mar. 7, 2024

(54) REGION EXTRACTION MODEL CREATION SUPPORT APPARATUS, METHOD FOR OPERATING REGION EXTRACTION MODEL CREATION SUPPORT APPARATUS, AND PROGRAM FOR OPERATING REGION EXTRACTION MODEL CREATION SUPPORT APPARATUS

(71) Applicant: FUJIFILM Corporation, Tokyo (JP)

(72) Inventor: Akimichi ICHINOSE, Tokyo (JP)

(73) Assignee: FUJIFILM Corporation, Tokyo (JP)

(21) Appl. No.: 18/509,243

(22) Filed: Nov. 14, 2023

Related U.S. Application Data
(63) Continuation of application No. PCT/JP2022/019543, filed on May 6, 2022.

(30) Foreign Application Priority Data
Jun. 1, 2021 (JP) 2021-092484

(51) Int. Cl. G06V 10/77; G06V 10/75; G06V 10/764; G06V 10/774

(52) U.S. Cl. CPC G06V 10/7715; G06V 10/751; G06V 10/764; G06V 10/774

(57) ABSTRACT

A learning unit uses a learning input image and local annotation data generated by locally giving labels to regions of classes in the learning input image as training data for a region extraction model. The learning unit directs the region extraction model to output a final feature amount map having element values related to probabilities of being the regions of the classes. Then, a sharpening process is performed on a probability distribution map that has been generated on the basis of the final feature amount map and that shows the probability for each class to obtain a processed probability distribution map. The learning unit calculates an average value of pixel values of the boundary image generated based on the processed probability distribution map as a boundary length loss, and updates the region extraction model in a direction in which the average value is reduced.

### CROSS-REFERENCE TO RELATED APPLICATIONS

This application is a continuation application of International Application No. PCT/JP2022/019543 filed on May 6, 2022, the disclosure of which is incorporated herein by reference in its entirety. Further, this application claims priority from Japanese Patent Application No. 2021-092484 filed on Jun. 1, 2021, the disclosure of which is incorporated herein by reference in its entirety.

### BACKGROUND

### 1. Technical Field

The technology of the present disclosure relates to a region extraction model creation support apparatus, a method for operating a region extraction model creation support apparatus, and a program for operating a region extraction model creation support apparatus.

### 2. Description of the Related Art

In a field of machine learning, for example, various region extraction models have been developed that recognize each lung lobe of lungs in a chest tomographic image captured by a computed tomography (CT) apparatus in units of pixels and extract regions of a plurality of classes which are in a subject to be recognized and whose boundaries are in contact with each other. In these region extraction models, training data is required in a learning phase. The training data is composed of a learning input image and annotation data. The annotation data is generated by manually giving labels corresponding to classes to the learning input image by an annotator. In a case of an example of the chest tomographic image, the annotation data is data generated by giving labels, such as a “right upper lobe”, a “right middle lobe”, a “left lower lobe”, and the “outside of a lung field” to the chest tomographic image as the learning input image.

The label is usually given to fill the entire region of the class. However, it takes a lot of time and effort to give the label. Therefore, in order to reduce the time and effort required to give the label, a technique has been proposed that roughly gives labels not to the entire region of a class, but to local parts in the region of the class at intervals to generate annotation data and trains a region extraction model using the generated annotation data (hereinafter, referred to as local annotation data).

The use of the local annotation data makes it possible to certainly reduce the time and effort required to give the label. However, the local annotation data is incomplete data as the training data, as compared to the annotation data in which the label is given to the entire region of the class. For this reason, the accuracy of extracting the region of the class in output data from the region extraction model is reduced. Specifically, in the output data, the boundary between the classes is jagged, or noise indicating the boundary between the classes is included in a portion which is not originally the boundary between the classes.

In Mehran Javanmardi, etc., “Unsupervised Total Variation Loss for Semi-supervised Deep Learning of Semantic Segmentation”, ECCV-16 (submission ID 868), 4 May 2016, in order to deal with the above-mentioned problem of the reduction in the extraction accuracy of the region of the class by the region extraction model due to the use of the local annotation data, the following process is performed in the learning phase of the region extraction model. That is, a boundary detection process using, for example, a Sobel filter is performed on a probability distribution map (a map indicating the probability of being the region of each class) in a stage before the output data to generate a boundary image from the probability distribution map. Then, an average value of pixel values of the boundary image is incorporated into the loss of the region extraction model to update the region extraction model in a direction in which the loss is reduced. Reducing the loss into which the average value of the pixel values of the boundary image has been incorporated leads to smoothing the jaggedness of the boundary between the classes in the output data using the probability distribution map or removing noise included in the portion that is not originally the boundary between the classes.

### SUMMARY

In Mehran Javanmardi, etc., “Unsupervised Total Variation Loss for Semi-supervised Deep Learning of Semantic Segmentation”, ECCV-16 (submission ID 868), 4 May 2016, in a case in which the boundary image generated from the probability distribution map is not a binary image of 0 and 1, but is an image having any value between 0 and 255, there is a concern that undesirable learning will be performed as illustrated in FIG. 28 as an example.

In FIG. 28, a star-shaped region 200 of a class is considered for the sake of simplification. In this case, it is desirable to perform learning that smooths the jaggedness of the boundary between the classes in the probability distribution map as represented by an upper circle 201. However, there is a concern that undesirable learning that smooths a change in the value of the class in the probability distribution map as represented by a lower star shape 202. The reason is that smoothing the jaggedness of the boundary between the classes in the probability distribution map (cutting the corners of the star of the region 200 to form the circle 201) and smoothing the change in the value of the class in the probability distribution map (gradually decreasing the value from the center of the region 200 toward the boundary while maintaining the star shape of the region 200) is equivalent to reducing the loss (in this case, the average value of the pixel values of the boundary image).

In a case in which the undesirable learning that smooths the change in the value of the class in the probability distribution map is performed, the smoothing of the jaggedness of the boundary between the classes in the probability distribution map is naturally suppressed. Therefore, there is still the problem that the extraction accuracy of the region of the class by the region extraction model is reduced due to the use of the local annotation data.

One embodiment of the technology of the present disclosure provides a region extraction model creation support apparatus, a method for operating a region extraction model creation support apparatus, and a program for operating a region extraction model creation support apparatus that can more suppress a reduction in extraction accuracy of a region of a class by a region extraction model due to use of local annotation data than the related art.

According to the present disclosure, there is provided a region extraction model creation support apparatus that supports creation of a region extraction model for extracting regions of a plurality of classes which are in a subject to be recognized in an image and whose boundaries are in contact with each other. The region extraction model creation support apparatus comprises: a processor; and a memory that is connected to or provided in the processor. The processor is configured to: use, as training data, a learning input image and local annotation data generated by locally giving labels to the regions of the classes in the learning input image; direct the region extraction model to output a final feature amount map having element values related to probabilities of being the regions of the classes; perform a sharpening process on the final feature amount map or a probability distribution map that has been generated on the basis of the final feature amount map and that shows the probability for each class; detect the boundary on the basis of a result of the sharpening process; and update the region extraction model in a direction in which a boundary length loss corresponding to a length of the boundary is reduced.

Preferably, the processor is configured to: direct the region extraction model to output learning output data obtained by extracting the regions of the classes in the learning input image; calculate a loss of the region extraction model according to a result of comparison between the local annotation data and the learning output data for local parts to which the labels have been given; add up the loss and the boundary length loss to obtain a first total loss; and update the region extraction model in a direction in which the first total loss is reduced.

Preferably, the processor is configured to: further add a size loss corresponding to sizes of the regions of the plurality of classes to the first total loss to obtain a second total loss; and update the region extraction model in a direction in which the second total loss is reduced.

Preferably, the sharpening process is a process of applying a softmax function with temperature having a temperature parameter equal to or less than 1 to the final feature amount map or the probability distribution map.

Preferably, the sharpening process is a process of applying a softargmax function to the final feature amount map or the probability distribution map.

Preferably, the sharpening process is a process of applying a sigmoid function having a gain equal to or greater than 1 to the final feature amount map or the probability distribution map.

Preferably, the boundary length loss is an average value of pixel values of a boundary image generated by detecting the boundary from the result of the sharpening process.

Preferably, the processor is configured to receive designation of a region from which the boundary is to be detected in the result of the sharpening process.

Preferably, the image is a medical image.

Preferably, the classes include a lung lobe.

According to the present disclosure, there is provided a method for operating a region extraction model creation support apparatus that supports creation of a region extraction model for extracting regions of a plurality of classes which are in a subject to be recognized in an image and whose boundaries are in contact with each other. The method comprises: using, as training data, a learning input image and local annotation data generated by locally giving labels to the regions of the classes in the learning input image; directing the region extraction model to output a final feature amount map having element values related to probabilities of being the regions of the classes; performing a sharpening process on the final feature amount map or a probability distribution map that has been generated on the basis of the final feature amount map and that shows the probability for each class; detecting the boundary on the basis of a result of the sharpening process; and updating the region extraction model in a direction in which a boundary length loss corresponding to a length of the boundary is reduced.

According to the present disclosure, there is provided a program for operating a region extraction model creation support apparatus that supports creation of a region extraction model for extracting regions of a plurality of classes which are in a subject to be recognized in an image and whose boundaries are in contact with each other. The program causes a computer to execute a process comprising: using, as training data, a learning input image and local annotation data generated by locally giving labels to the regions of the classes in the learning input image; directing the region extraction model to output a final feature amount map having element values related to probabilities of being the regions of the classes; performing a sharpening process on the final feature amount map or a probability distribution map that has been generated on the basis of the final feature amount map and that shows the probability for each class; detecting the boundary on the basis of a result of the sharpening process; and updating the region extraction model in a direction in which a boundary length loss corresponding to a length of the boundary is reduced.

According to the technology of the present disclosure, it is possible to provide a region extraction model creation support apparatus, a method for operating a region extraction model creation support apparatus, and a program for operating a region extraction model creation support apparatus that can more suppress a reduction in extraction accuracy of a region of a class by a region extraction model due to use of local annotation data than the related art.

### BRIEF DESCRIPTION OF THE DRAWINGS

Exemplary embodiments according to the technique of the present disclosure will be described in detail based on the following figures, wherein:

FIG. 1 is a diagram illustrating a region extraction model creation support server and an annotator terminal;

FIG. 2 is a diagram illustrating a learning input image and local annotation data;

FIG. 3 is a diagram illustrating an aspect in which training data is transmitted from the annotator terminal to the region extraction model creation support server;

FIG. 4 is a block diagram illustrating a computer constituting the region extraction model creation support server;

FIG. 5 is a block diagram illustrating processing units of a CPU of the region extraction model creation support server;

FIG. 6 is a block diagram illustrating a detailed configuration of a region extraction model;

FIG. 7 is a diagram illustrating an element of a probability distribution map;

FIG. 8 is a diagram illustrating a sharpening process;

FIG. 9 is a diagram illustrating details of the sharpening process;

FIG. 10 is a diagram illustrating a boundary detection process;

FIG. 11 is a diagram illustrating an average value calculation process;

FIG. 12 is a diagram illustrating a loss calculation process;

FIG. 13 is a diagram illustrating an update setting process;

FIG. 14 is a diagram illustrating an example of a change in a boundary image from an early stage of learning to an end stage of learning;

FIG. 15 is a flowchart illustrating a processing procedure of the region extraction model creation support server;

FIG. 16 is a diagram illustrating an aspect in which an input image in which a region of each class is unknown is input to a trained region extraction model and output data in which a label has been given to the region of each class in the input image is output from the trained region extraction model;

FIG. 17 is a diagram illustrating a lung lobe size dispersion calculation process;

FIG. 18 is a diagram illustrating an update setting process according to a second embodiment;

FIG. 19 is a diagram illustrating learning output data in a case of being trapped into a local solution;

FIG. 20 is a diagram illustrating local annotation data according to a third embodiment;

FIG. 21 is a diagram illustrating an aspect in which a learning input image is input to a lung field extraction model and lung field extraction data is output from the lung field extraction model;

FIG. 22 is a diagram illustrating a boundary detection process according to a third embodiment;

FIG. 23 is a diagram illustrating a sharpening process according to a fourth embodiment;

FIG. 24 is a diagram illustrating a sharpening process according to a fifth embodiment;

FIG. 25 is a diagram illustrating another example of the sharpening process according to the fifth embodiment;

FIG. 26 is a diagram illustrating a sharpening process according to a sixth embodiment;

FIG. 27 is a diagram illustrating another example of the sharpening process according to the sixth embodiment; and

FIG. 28 is a diagram illustrating problems of the related art.

### DETAILED DESCRIPTION

### First Embodiment

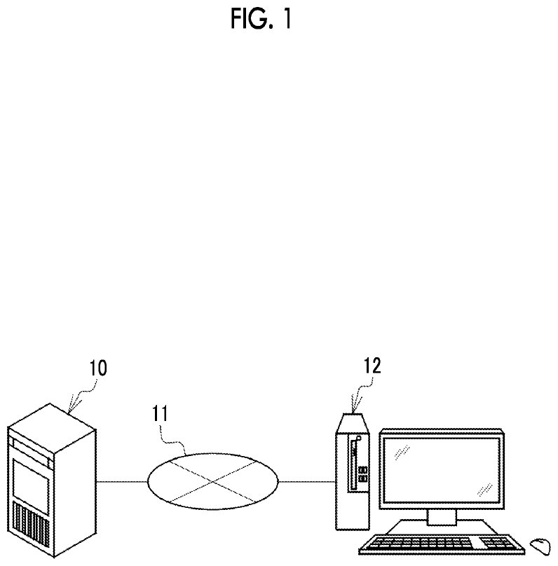

For example, as illustrated in FIG. 1, a region extraction model creation support server (hereinafter, abbreviated to a support server) 10 is connected to an annotator terminal 12 through a network 11 such that it can communicate with the annotator terminal 12. The network 11 is, for example, a wide area network (WAN) such as the Internet or a public communication network. In addition, only one annotator terminal 12 is illustrated. However, a plurality of annotator terminals 12 are actually provided.

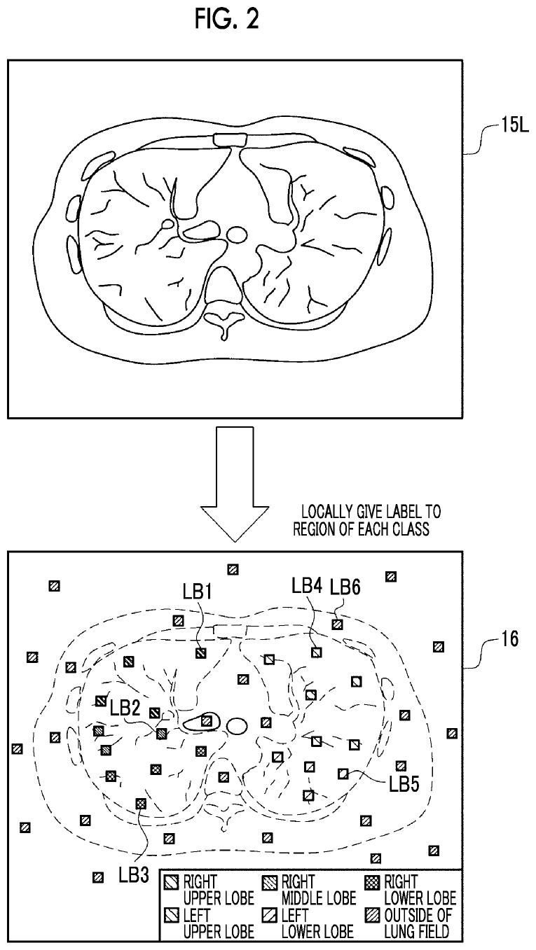

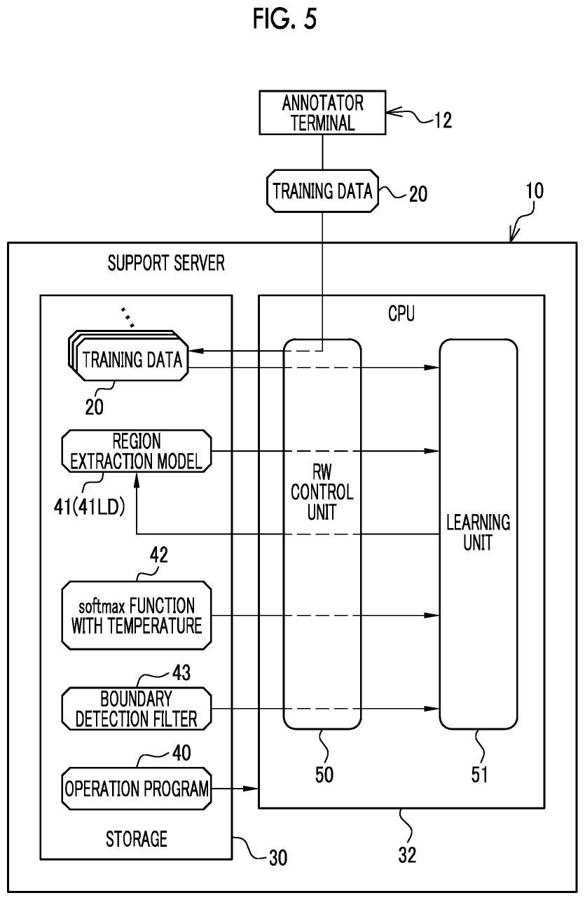

The support server 10 is, for example, a server computer or a workstation. The support server 10 supports the creation of a region extraction model 41 (see FIG. 5) for extracting a plurality of regions of classes which are in a subject to be recognized in an image and whose boundaries are in contact with each other. That is, the support server 10 is an example of a “region extraction model creation support apparatus” according to the technology of the present disclosure. The annotator terminal 12 is a terminal that is operated by an annotator who generates local annotation data 16 (see FIG. 2). The annotator terminal 12 is, for example, a personal computer or a tablet terminal.

For example, as illustrated in FIG. 2, the annotator terminal 12 displays a learning input image 15L on a display. In this example, the learning input image 15L is a tomographic image obtained by imaging a chest of a patient who is suspected to have a lung disease, such as pneumonia or lung cancer, using a computed tomography (CT) apparatus. As is well known, the CT apparatus performs radiography on a patient at different projection angles to acquire a plurality of projection data items, reconstructs the plurality of acquired projection data items, and outputs a tomographic image. The tomographic image is voxel data indicating a three-dimensional shape of an internal structure of the patient. FIG. 2 illustrates a tomographic image at a slice position in an axial cross section. In this example, the tomographic image is an image that mainly includes the lung of the patient. The learning input image 15L is an example of a “medical image” according to the technology of the present disclosure.

The annotator operates an input device of the annotator terminal 12 to locally give a square label LB having a size of the square of several pixels to the region of each class in the learning input image 15L. Specifically, the annotator gives a label LB1 to a local part of a region that is considered to be a right upper lobe, gives a label LB2 to a local part of a region that is considered to be a right middle lobe, and gives a label LB3 to a local part of a region that is considered to be a right lower lobe. In addition, the annotator gives a label LB4 to a local part of a region that is considered to be a left upper lobe and gives a label LB5 to a local part of a region that is considered to be a left lower lobe. Further, the annotator gives a label LB6 to a local part of a region that is considered to be the outside of a lung field. As a result, local annotation data 16 is generated. As can be seen from this description, the classes in this example are the lung lobes of the right upper lobe, the right middle lobe, the right lower lobe, the left upper lobe, and the left lower lobe, and the outside of the lung field. That is, the classes include the lung lobes. In addition, the annotator gives the labels LB to each slice position of the tomographic image to generate the local annotation data 16.

Further, in FIG. 2, for ease of understanding, a human body structure is drawn in the local annotation data 16. However, the actual local annotation data 16 does not include data of the human body structure and includes only the data of the given labels LB. More specifically, the local annotation data 16 is data in which a set of the type of the label LB and the coordinates of the position of a pixel of the learning input image 15L having the label LB given thereto has been registered.

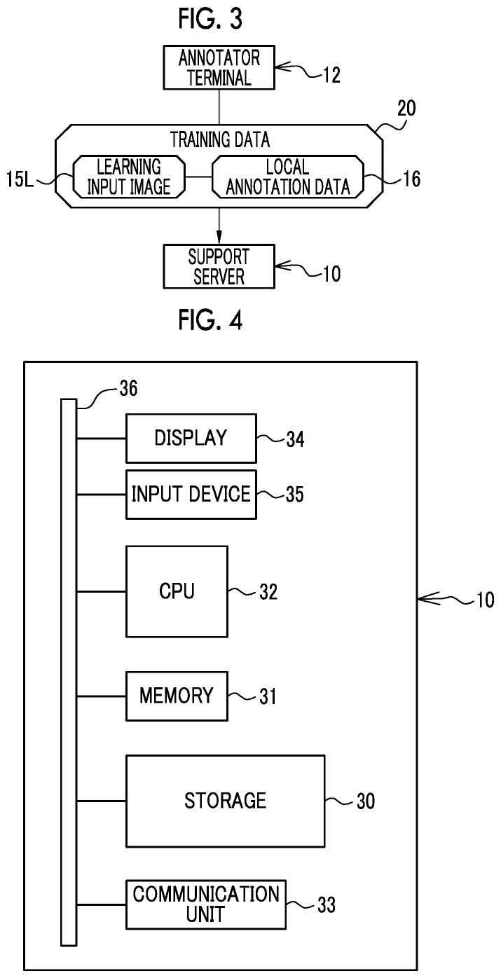

For example, as illustrated in FIG. 3, the annotator terminal 12 transmits a set of the learning input image 15L and the local annotation data 16 as training data 20 to the support server 10. The support server 10 receives the training data 20 from the annotator terminal 12. The support server 10 trains the region extraction model 41 using the received training data 20.

For example, as illustrated in FIG. 4, a computer that constitutes the support server 10 comprises a storage 30, a memory 31, a central processing unit (CPU) 32, a communication unit 33, a display 34, and an input device 35. These components are connected to each other through a bus line 36.

The storage 30 is a hard disk drive that is provided in the computer constituting the support server 10 or that is connected to the computer through a cable or a network. Alternatively, the storage 30 is a disk array in which a plurality of hard disk drives are connected in series. The storage 30 stores a control program, such as an operating system, various application programs, various types of data associated with these programs, and the like. In addition, a solid state drive may be used instead of the hard disk drive.

The memory 31 is a work memory for the CPU 32 to perform processes. The CPU 32 loads the program stored in the storage 30 to the memory 31 and performs a process corresponding to the program. Therefore, the CPU 32 controls the overall operation of each unit of the computer. In addition, the CPU 32 is an example of a “processor” according to the technology of the present disclosure. Further, the memory 31 may be provided in the CPU 32.

The communication unit 33 is a network interface that controls the transmission of various types of information through the network 11 or the like. The display 34 displays various screens. The various screens have operation functions by a graphical user interface (GUI). The computer constituting the support server 10 receives an input of an operation instruction from the input device 35 through the various screens. The input device 35 is, for example, a keyboard, a mouse, and a touch panel.

For example, as illustrated in FIG. 5, an operation program 40 is stored in the storage 30 of the support server 10. The operation program 40 is an application program that causes the computer constituting the support server 10 to function as a “region extraction model creation support apparatus” according to the technology of the present disclosure. That is, the operation program 40 is an example of a “program for operating a region extraction model creation support apparatus” according to the technology of the present disclosure. The storage 30 stores a plurality of training data items 20 from the annotator terminal 12, the region extraction model 41, a softmax function 42 with temperature, and a boundary detection filter 43 in addition to the operation program 40.

In a case in which the operation program 40 is started, the CPU 32 of the support server 10 functions as a read and write (hereinafter, abbreviated to RW) control unit 50 and a learning unit 51 in cooperation with, for example, the memory 31.

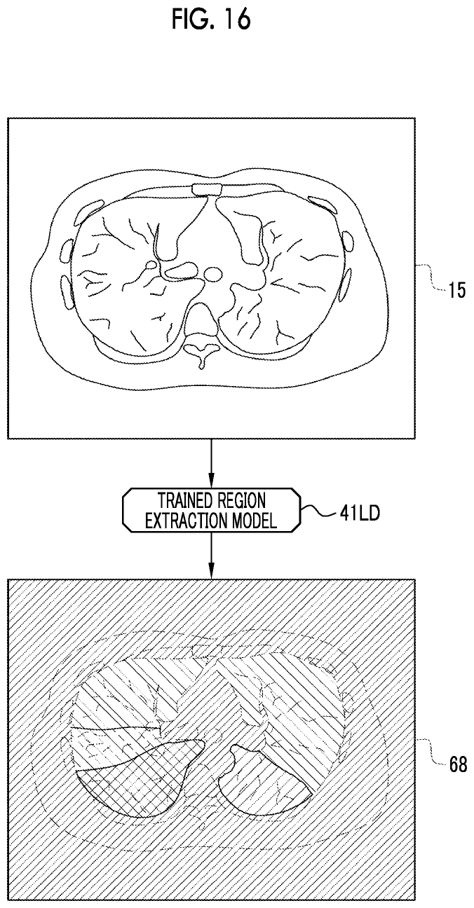

The RW control unit 50 controls the storage of various types of data in the storage 30 and the reading of various types of data from the storage 30. For example, the RW control unit 50 stores the training data 20 from the annotator terminal 12 in the storage 30. In addition, the RW control unit 50 reads the region extraction model 41, the softmax function 42 with temperature, and the boundary detection filter 43 from the storage 30 and outputs the read region extraction model 41, softmax function 42 with temperature, and boundary detection filter 43 to the learning unit 51. Further, the RW control unit 50 reads the training data 20 from the storage 30 and outputs the read training data 20 to the learning unit 51. Furthermore, the RW control unit 50 stores a region extraction model (hereinafter, referred to as a trained region extraction model) 41LD (see FIG. 16), which has been trained, from the learning unit 51 in the storage 30.

The region extraction model 41 is a machine learning model for extracting each of the lung lobes and a region outside the lung field. The region extraction model 41 is constructed by a convolutional neural network (CNN) such as Residual Networks (ResNet) or U-Shaped Networks (U-Net). The learning unit 51 trains the region extraction model 41 using the training data 20, the softmax function 42 with temperature, and the boundary detection filter 43.

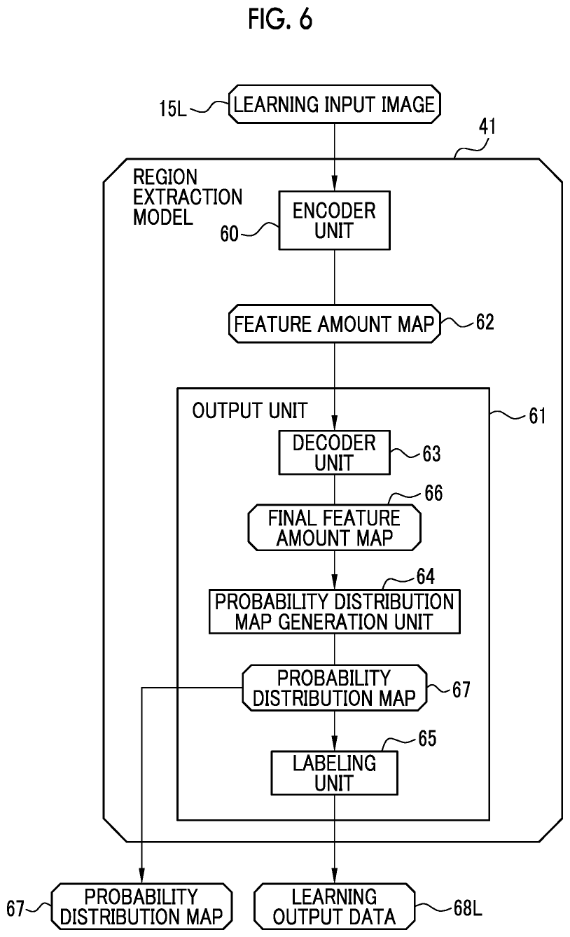

For example, as illustrated in FIG. 6, the learning unit 51 inputs the learning input image 15L in the training data 20 to the region extraction model 41. The region extraction model 41 is composed of an encoder unit 60 and an output unit 61. The encoder unit 60 includes a plurality of convolutional layers that perform a convolution process using a filter and a plurality of pooling layers that perform a pooling process of calculating a local statistic of the data subjected to the convolution process to reduce the amount of data subjected to the convolution process. The encoder unit 60 converts the learning input image 15L into a feature amount map 62. The encoder unit 60 outputs the feature amount map 62 to the output unit 61. In addition, the encoder unit 60 also performs, for example, skip layer processing for delivering the data subjected to the convolution process to the output unit 61, which is not illustrated.

The output unit 61 includes a decoder unit 63, a probability distribution map generation unit 64, and a labeling unit 65. The decoder unit 63 performs an upsampling process of enlarging the feature amount map 62 to obtain an enlarged feature amount map. The decoder unit 63 also performs a convolution process simultaneously with the upsampling process. Further, the decoder unit 63 performs a merging process of merging the enlarged feature amount map with the data subjected to the convolution process which has been delivered from the encoder unit 60 by the skip layer processing. The decoder unit 63 further performs the convolution process after the merging process. The decoder unit 63 converts the feature amount map 62 into a final feature amount map 66 through these various processes.

The final feature amount map 66 is also referred to as logits and has elements that are in one-to-one correspondence with the pixels of the learning input image 15L. Each element of the final feature amount map 66 has an element value related to each class. The decoder unit 63 outputs the final feature amount map 66 to the probability distribution map generation unit 64.

The probability distribution map generation unit 64 generates a probability distribution map 67 from the final feature amount map 66 using a known activation function such as a softmax function. The probability distribution map generation unit 64 outputs the probability distribution map 67 to the labeling unit 65.

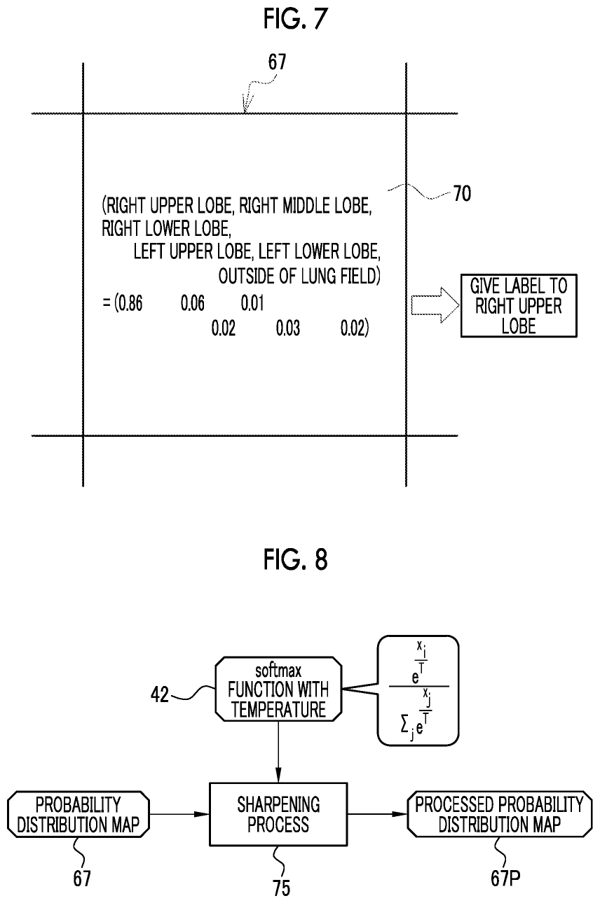

For example, as illustrated in FIG. 7, similarly to the final feature amount map 66, the probability distribution map 67 is data which has elements 70 that are in one-to-one correspondence with the pixels of the learning input image 15L and in which a probability that each element will be the region of each class has been registered as an element value of each element 70. FIG. 7 illustrates a case in which the probability that the element 70 will be the region of the right upper lobe is 86% (0.86) and the probabilities that the element 70 will be the regions of the right middle lobe, the right lower lobe, the left upper lobe, the left lower lobe, and the outside of the lung field are 6%, 1%, 2%, 3%, and 2%, respectively. All of the probabilities that the element will be the region of each class add up to 100%.

The labeling unit 65 gives the label LB of the class having the maximum probability to each element 70 of the probability distribution map 67. Therefore, in the example illustrated in FIG. 7, the labeling unit 65 gives the label LB1 of the right upper lobe having the maximum probability of 86%. The labeling unit 65 gives the label LB to each element 70 of the probability distribution map 67 in this way and outputs learning output data 68L. The learning output data 68L is data in which any one of the labels LB1 to LB6 of the six classes has been given to each pixel of the learning input image 15L.

For example, as illustrated in FIG. 8, the learning unit 51 performs a sharpening process 75 on the probability distribution map 67 output from the region extraction model 41 to convert the probability distribution map 67 into a processed probability distribution map 67P. The sharpening process 75 is a process of applying the softmax function 42 with temperature to the probability distribution map 67. The softmax function 42 with temperature is a function that is shown in a balloon of FIG. 8 and is represented by the following Expression (1). In addition, x is the probability that the element will be each class in the probability distribution map 67. Further, i and j indicate class numbers, and i indicates the class number for which the probability is calculated. Further, T is a temperature parameter and is set to a sufficiently small value that is equal or less than 1 (T≤1).

$$\begin{matrix}[ Expression 1 ] & \\ \frac{{e}^{\frac{{x}_{i}}{T}}}{{\sum }_{j} {e}^{\frac{{x}_{j}}{T}}} & ( 1 )\end{matrix}$$

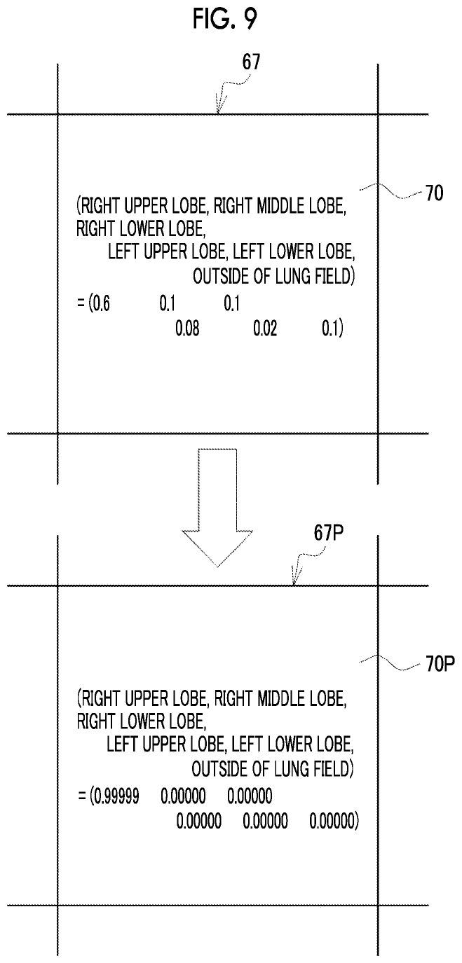

For example, as illustrated in FIG. 9, the softmax function 42 with temperature is applied to convert the probabilities that the element 70 will be each class in the probability distribution map 67 such that the probability that an element 70P of the processed probability distribution map 67P will be a certain class is 99.999% (0.99999) and the probabilities that the element 70P will be all of the other classes are 0% (0.00000). That is, while the probability distribution map 67 is data having any value between 0 and 1, the processed probability distribution map 67P is data having both extreme values such as two values of 0.99999 and 0.00000. The processed probability distribution map 67P is an example of a “result of a sharpening process” according to the technology of the present disclosure.

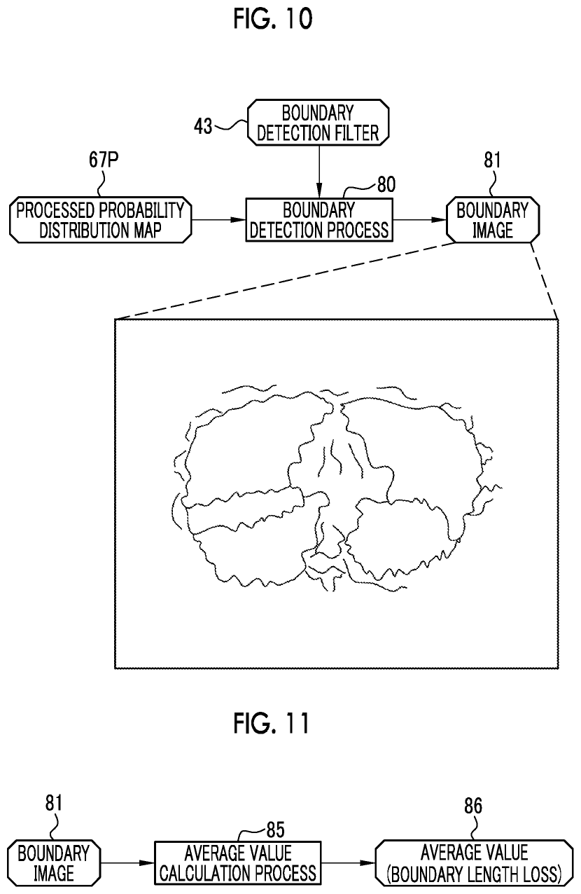

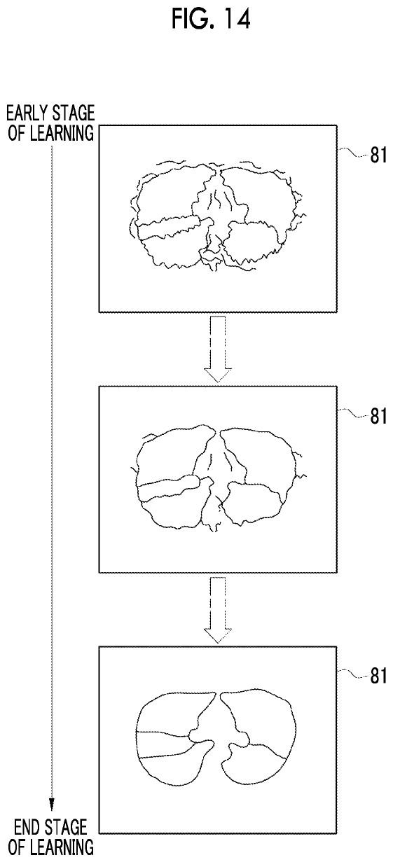

For example, as illustrated in FIG. 10, the learning unit 51 performs a boundary detection process 80 of applying the boundary detection filter 43 to the processed probability distribution map 67P to generate a boundary image 81. The boundary detection filter 43 is, for example, a Prewitt filter or a Sobel filter. The boundary image 81 is an image in which a pixel value of a pixel in a portion that is considered to be the boundary of the region of each class is remarkably large. Further, in FIG. 10, the boundary image 81 is drawn as a two-dimensional image. However, the actual boundary image 81 is a three-dimensional image. The same is applied to FIG. 14 and the like which will be described below.

For example, as illustrated in FIG. 11, the learning unit 51 performs an average value calculation process 85. The average value calculation process 85 is a process of calculating an average value 86 of pixel values of the boundary image 81. The average value 86 is larger as the number of portions considered to be the boundaries in the boundary image 81 is larger. Therefore, in a case in which a portion that is considered to be the boundary between the classes is jagged or a portion that is originally not the boundary between the classes includes noise, the average value 86 is a large value. The average value 86 is an example of a “boundary length loss” according to the technology of the present disclosure.

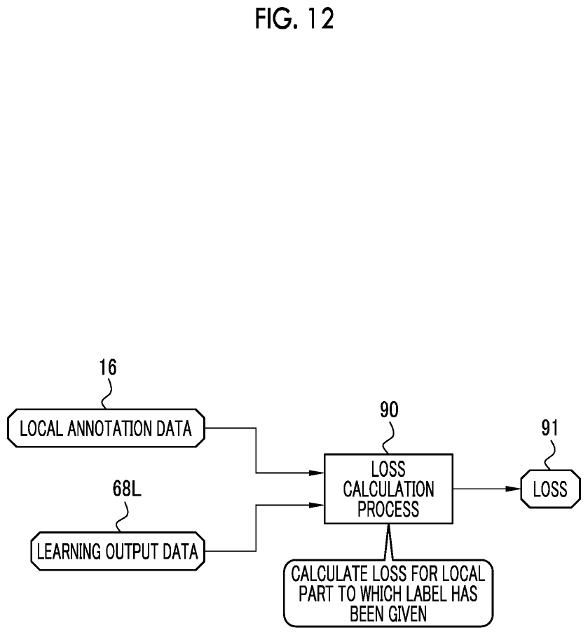

For example, as illustrated in FIG. 12, the learning unit 51 performs a loss calculation process 90. The loss calculation process 90 is a process of calculating a loss 91 of the region extraction model 41 corresponding to the result of comparison between the local annotation data 16 and the learning output data 68L. The loss 91 is a cross entropy error. In the loss calculation process 90, the loss 91 is calculated only for the local part to which the label LB has been given. In a case in which the label LB given to the local annotation data 16 and the label LB given to the learning output data 68L are different from each other, the loss 91 is large. On the contrary, in a case in which the label LB given to the local annotation data 16 and the label LB given to the learning output data 68L are matched with each other, the loss 91 is small.

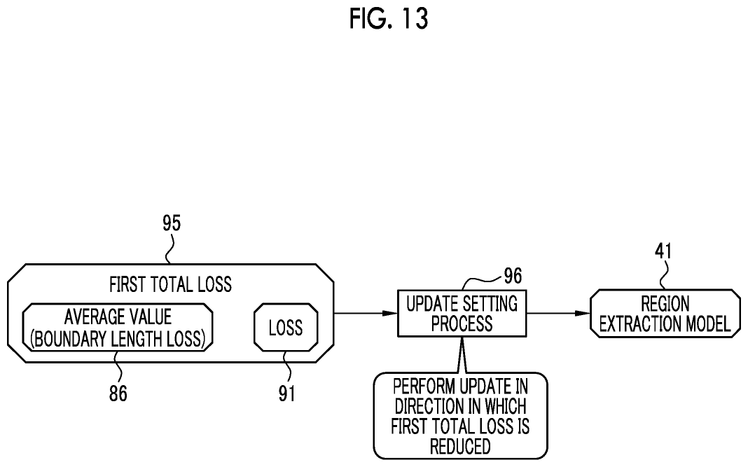

For example, as illustrated in FIG. 13, the learning unit 51 adds up the loss 91 of the region extraction model 41 and the average value 86 as the boundary length loss to obtain a first total loss 95. Then, the learning unit 51 performs an update setting process 96. The update setting process 96 is a process of updating, for example, a value of a coefficient of the filter of the region extraction model 41 in a direction in which the first total loss 95 is reduced, using a well-known backpropagation method. The extraction accuracy of the region extraction model 41 is more improved than before by the update setting process 96.

FIG. 14 illustrates an example of a change of the boundary image 81 from an early stage of learning to an end stage of learning. In the boundary image 81 at the early stage of learning, a portion that is considered to be the boundary between the classes is quite jagged, and noise is included in a portion that is not originally the boundary between the classes. However, as the learning progresses, the jaggedness of the portion that is considered to be the boundary between the classes is reduced, and the noise is also reduced. Finally, in the boundary image 81 at the end stage of learning, the jaggedness of the portion that is considered to be the boundary between the classes is substantially removed, and the noise is substantially removed.

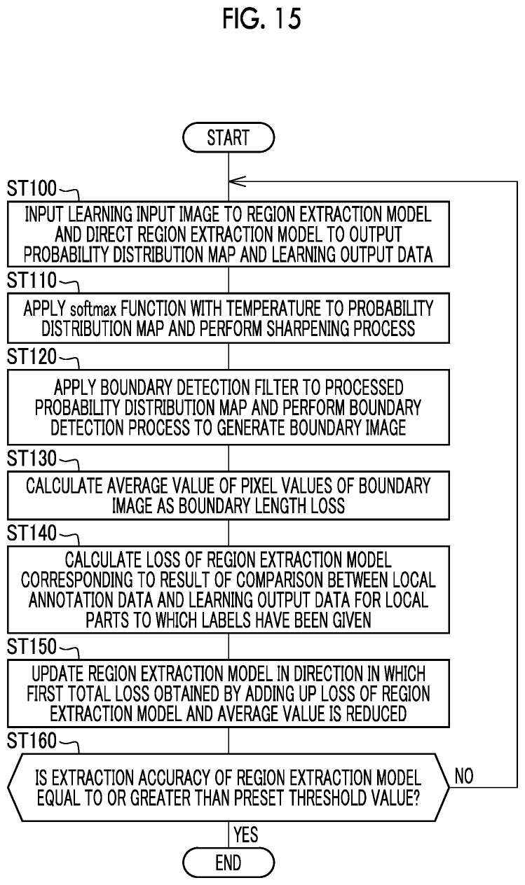

Next, the operation of the above-described configuration will be described with reference to, for example, a flowchart illustrated in FIG. 15. In a case in which the operation program 40 is started, the CPU 32 of the support server 10 functions as the RW control unit 50 and the learning unit 51 as illustrated in FIG. 5.

The RW control unit 50 reads the region extraction model 41, the softmax function 42 with temperature, and the boundary detection filter 43 from the storage 30 and outputs them to the learning unit 51. Further, the RW control unit 50 reads one training data item 20 from the storage 30 and outputs the read training data item 20 to the learning unit 51.

In the learning unit 51, the learning input image 15L is input to the region extraction model 41 as illustrated in FIG. 6. Then, the probability distribution map 67 and the learning output data 68L are output from the region extraction model 41 (Step ST100).

As illustrated in FIG. 8, the softmax function 42 with temperature is applied to the probability distribution map 67, and the sharpening process 75 is performed to convert the probability distribution map 67 into the processed probability distribution map 67P (Step ST110). Then, as illustrated in FIG. 10, the boundary detection filter 43 is applied to the processed probability distribution map 67P, and the boundary detection process 80 is performed to generate the boundary image 81 (Step ST120).

The average value calculation process 85 is performed to calculate the average value 86 of the pixel values of the boundary image 81 as the boundary length loss as illustrated in FIG. 11 (Step ST130). Further, as illustrated in FIG. 12, the loss calculation process 90 is performed to calculate the loss 91 of the region extraction model 41 corresponding to the result of the comparison between the local annotation data 16 and the learning output data 68L for the local parts to which the labels LB have been given (Step ST140).

As illustrated in FIG. 13, the update setting process 96 is performed to update the region extraction model 41 in the direction in which the first total loss 95 obtained by adding up the loss 91 of the region extraction model 41 and the average value 86 is reduced (Step ST150). The series of processes in Steps ST100 to ST150 is repeatedly performed in a case in which the extraction accuracy of the region extraction model 41 is not equal to or greater than a preset threshold value (NO in Step ST160).

In a case in which the extraction accuracy of the region extraction model 41 is equal to or greater than the threshold value (YES in Step ST160), the region extraction model 41 is stored as the trained region extraction model 41LD in the storage 30 by the RW control unit 50.

For example, as illustrated in FIG. 16, an input image 15 in which the region of each class is unknown is input to the trained region extraction model 41LD. The trained region extraction model 41LD outputs output data 68 in which the labels LB have been given to the regions of each class in the input image 15. The regions of each class in the output data 68 have boundaries that are in contact with each other.

As described above, the CPU 32 of the support server 10 comprises the learning unit 51. The learning unit 51 uses the learning input image 15L and the local annotation data 16 generated by locally giving the label LB to the region of each class in the learning input image 15L as the training data 20 for the region extraction model 41. The learning unit 51 directs the region extraction model 41 to output the final feature amount map 66 having the element values related to the probabilities that each element will be the region of each class. Then, the sharpening process 75 is performed on the probability distribution map 67, which shows the probability for each class and has been generated on the basis of the final feature amount map 66, to obtain the processed probability distribution map 67P. The learning unit 51 generates the boundary image 81 on the basis of the processed probability distribution map 67P that is the result of the sharpening process. The learning unit 51 calculates the average value 86 of the pixel values of the boundary image 81 as the boundary length loss and updates the region extraction model 41 in the direction in which the average value 86 is reduced.

The processed probability distribution map 67P is, for example, data having both extreme values such as 0.99999 and 0.00000. Therefore, the boundary image 81 generated on the basis of the processed probability distribution map 67P does not have, for example, any value between 0 and 255, but has only a few values. Therefore, as in Mehran Javanmardi, etc., “Unsupervised Total Variation Loss for Semi-supervised Deep Learning of Semantic Segmentation”, ECCV-16 (submission ID 868), 4 May 2016, the concern that undesirable learning which smooths a change in the value of the class in the probability distribution map 67 will be performed is suppressed. From the above, according to the technology of the present disclosure, it is possible to more suppress a reduction in the extraction accuracy of the region of the class by the region extraction model 41 due to the use of the local annotation data 16 than the related art.

Reducing the average value 86 as the boundary length loss leads to smoothing the jaggedness of the boundary between the classes in the probability distribution map 67 and thus in the learning output data 68L and removing noise indicating the boundary between the classes in the portion that is not originally the boundary between the classes. Therefore, as illustrated in FIG. 14, at the end stage of learning, the jaggedness of the portion that is considered to be the boundary between the classes is substantially removed and noise is also substantially removed. As a result, it is possible to extract the region of the class with higher accuracy.

The learning unit 51 directs the region extraction model 41 to output the learning output data 68L obtained by extracting the region of the class in the learning input image 15L. Then, the loss 91 of the region extraction model 41 corresponding to the result of the comparison between the local annotation data 16 and the learning output data 68L for the local parts to which the labels LB have been given is calculated. The learning unit 51 adds up the loss 91 and the average value 86 as the boundary length loss to obtain the first total loss 95 and updates the region extraction model 41 in the direction in which the first total loss 95 is reduced.

In a case in which the region extraction model 41 is updated in the direction in which the first total loss 95 obtained by incorporating the average value 86 as the boundary length loss into the loss 91 of the region extraction model 41 is reduced, learning for removing the jaggedness of the portion that is considered to be the boundary between the classes and for removing noise can be performed as part of learning for reducing the loss 91 of the region extraction model 41.

The sharpening process 75 is a process of applying the softmax function 42 with temperature to the probability distribution map 67. Therefore, it is possible to easily convert the probability distribution map 67 into the processed probability distribution map 67P. In addition, it is possible to smoothly perform the update setting process 96 using the backpropagation method.

The boundary length loss is the average value 86 of the pixel values of the boundary image 81 generated by detecting the boundary from the processed probability distribution map 67P. Therefore, it is possible to easily calculate the boundary length loss. In addition, instead of the average value 86, a sum of the pixel values of the boundary image 81 may be used as the boundary length loss.

In this example, the tomographic image in which the lung of the patient is mainly included is used as the learning input image 15L. Further, in this example, the classes are the lung lobes of the right upper lobe, the right middle lobe, the right lower lobe, the left upper lobe, and the left lower lobe and the outside of the lung field and include the lung lobes.

In the medical field, there is a strong demand to extract the region of each part of the organs included in a medical image with the region extraction model 41 and to present a result of the extraction to the doctor, thereby supporting diagnosis of the doctor. In addition, a lung disease, such as pneumonia which has been increasing in recent years as a cause of death of an elderly person or lung cancer which is listed as the top cause of cancer of a male, has attracted attention. Therefore, there is also a strong demand to recognize the lung lobes related to the lung disease with a certain degree of accuracy without taking time and effort. In the technology of the present disclosure, the image is a medical image, and the lung lobes are included in the classes. Therefore, it is possible to meet these demands.

### Second Embodiment

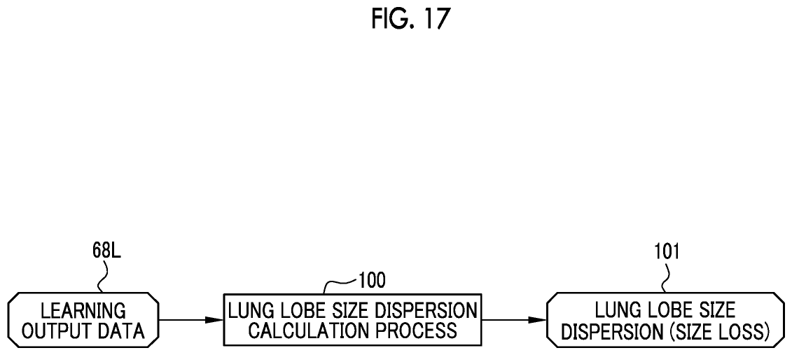

For example, as illustrated in FIG. 17, in this embodiment, the learning unit 51 performs a lung lobe size dispersion calculation process 100 of calculating a dispersion (hereinafter, referred to as a lung lobe size dispersion) 101 of the size of the region of each lung lobe. First, the learning unit 51 calculates the total number of pixels, to which the labels LB1 to LB5 of each lung lobe have been given, from the learning output data 68L. The total number of pixels to which the labels LB1 to LB5 of each lung lobe have been given indicates the size of the region of each lung lobe. The learning unit 51 calculates the lung lobe size dispersion 101 from the calculated total number of pixels. The lung lobe size dispersion 101 has a larger value as a variation in the size of each lung lobe is larger. The lung lobe size dispersion 101 is an example of a “size loss” according to the technology of the present disclosure.

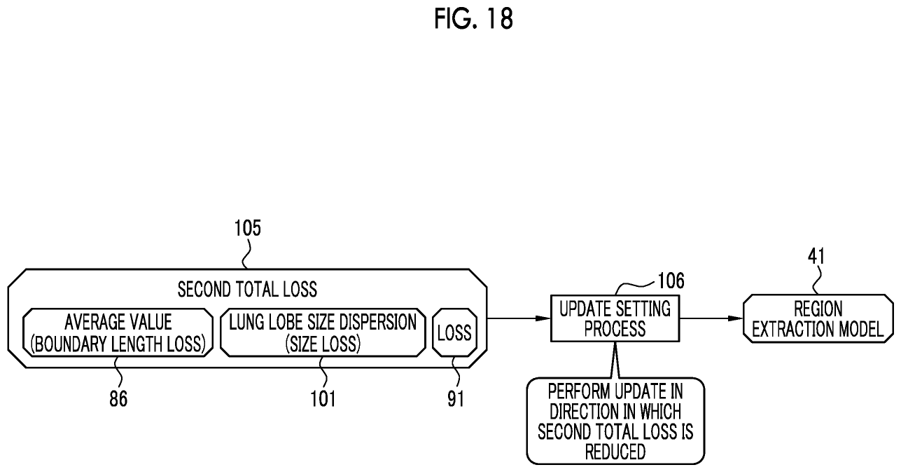

For example, as illustrated in FIG. 18, the learning unit 51 can add up the loss 91 of the region extraction model 41, the average value 86 as the boundary length loss, and the lung lobe size dispersion 101 as the size loss to obtain a second total loss 105. The second total loss 105 is obtained by adding the lung lobe size dispersion 101 as the size loss to the first total loss 95 according to the first embodiment which is the sum of the loss 91 of the region extraction model 41 and the average value 86 as the boundary length loss.

The learning unit 51 performs an update setting process 106. The update setting process 106 is a process of updating, for example, the value of the coefficient of the filter of the region extraction model 41 in a direction in which the second total loss 105 is reduced, using a well-known backpropagation method, similarly to the update setting process 96 according to the first embodiment. The extraction accuracy of the region extraction model 41 is more improved than before by the update setting process 106.

In a case in which the update setting process 96 of updating the region extraction model 41 in the direction in which the average value 86 as the boundary length loss is reduced is excessively performed, there is a concern of being trapped into a local solution illustrated in FIG. 19 as an example. That is, there is a concern that the learning output data 68L, in which the label LB5 has been given to only the local part to which the label LB5 of the left lower lobe has been given in the local annotation data 16, will be output. This is because the region to which the label LB5 has been given in the learning output data 68L has a short boundary length, that is, has a small average value 86. Further, since the region to which the label LB5 has been given in the learning output data 68L is substantially the same as the region to which the label LB5 has been given in the local annotation data 16, the loss 91 of the region extraction model 41 is also small. Therefore, even in a case in which the learning output data 68L is output, it is misunderstood that the learning is going well.

Since the lung lobes are known to have substantially the same size, the local solution illustrated in FIG. 19 is not actually obtained. However, for the above reasons, there is a concern of being trapped into the local solution illustrated in FIG. 19. Therefore, in the second embodiment, the learning unit 51 further adds the lung lobe size dispersion 101 as the size loss corresponding to the size of the region of each lung lobe to the first total loss 95 to obtain the second total loss 105. Further, the learning unit 51 updates the region extraction model 41 in the direction in which the second total loss 105 is reduced.

Reducing the second total loss 105 means reducing the lung lobe size dispersion 101. Reducing the lung lobe size dispersion 101 leads to making the sizes of each lung lobe in the learning output data 68L substantially the same. That is, it is possible to remove the concern of being trapped into the local solution as in the learning output data 68L illustrated in FIG. 19. In addition, instead of the lung lobe size dispersion 101, a standard deviation of the sizes of each lung lobe may be used as the size loss.

### Third Embodiment

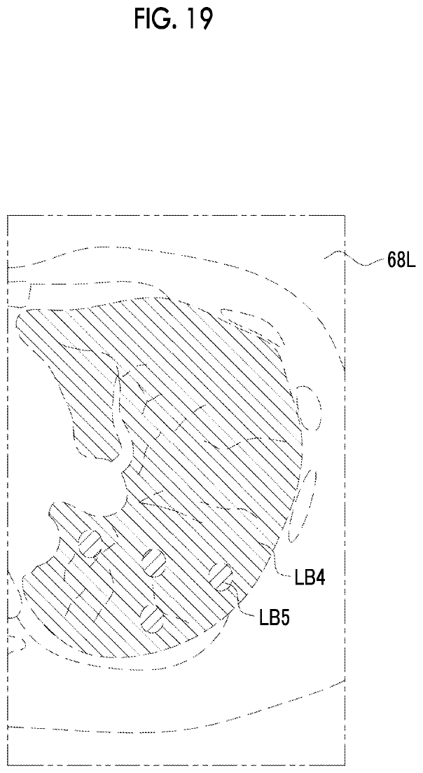

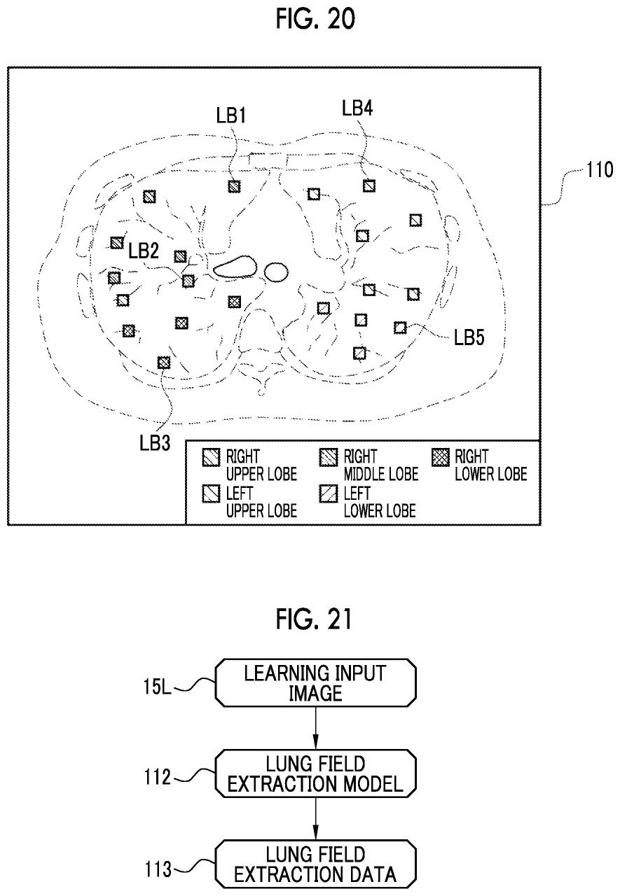

For example, as illustrated in FIG. 20, in this embodiment, local annotation data 110 is used in which the labels LB1 to LB5 of the lung lobes have been given, but the label LB6 of the outside of the lung field has not been given.

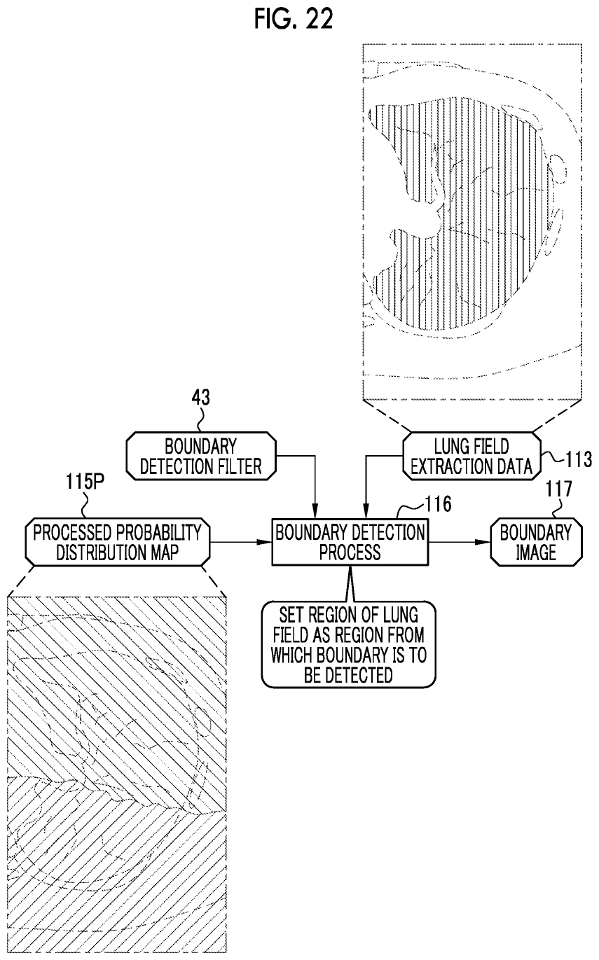

For example, as illustrated in FIG. 21, the learning unit 51 inputs the learning input image 15L to a lung field extraction model 112 prior to the training of the region extraction model 41. The lung field extraction model 112 is a trained machine learning model that is prepared separately from the region extraction model 41. The lung field extraction model 112 extracts the region of the lung field included in the learning input image 15L and outputs lung field extraction data 113 (also see FIG. 22) in which a label has been given to the extracted region of the lung field.

For example, as illustrated in FIG. 22, since the local annotation data 110 in which the label LB6 of the outside of the lung field has not been given is used, a processed probability distribution map 115P according to this embodiment is data in which the lung lobes are distinguished from each other, but the outside of the lung field is not distinguished. The learning unit 51 applies the boundary detection filter 43 to the processed probability distribution map 115P and performs a boundary detection process 116 to generate a boundary image 117.

In the generation of the boundary image 117, the learning unit 51 sets the region of the lung field indicated by the lung field extraction data 113 as a region from which the boundary is to be detected. That is, the learning unit 51 directs the lung field extraction model 112 to output the lung field extraction data 113 and receives the designation of the region from which the boundary is to be detected in the processed probability distribution map 115P. Therefore, as compared to the first embodiment in which the boundary detection process 80 is performed on the entire processed probability distribution map 67P including the outside of the lung field, it is possible to reduce the processing load of the boundary detection process 116 and to shorten the processing time of the boundary detection process 116. In addition, the label LB 6 of the outside of the lung field may not be given. Therefore, it is possible to further reduce the time and effort required to give the label LB. Further, the designation of the region from which the boundary is to be detected in the processed probability distribution map 115P by the user may be received.

### Fourth Embodiment

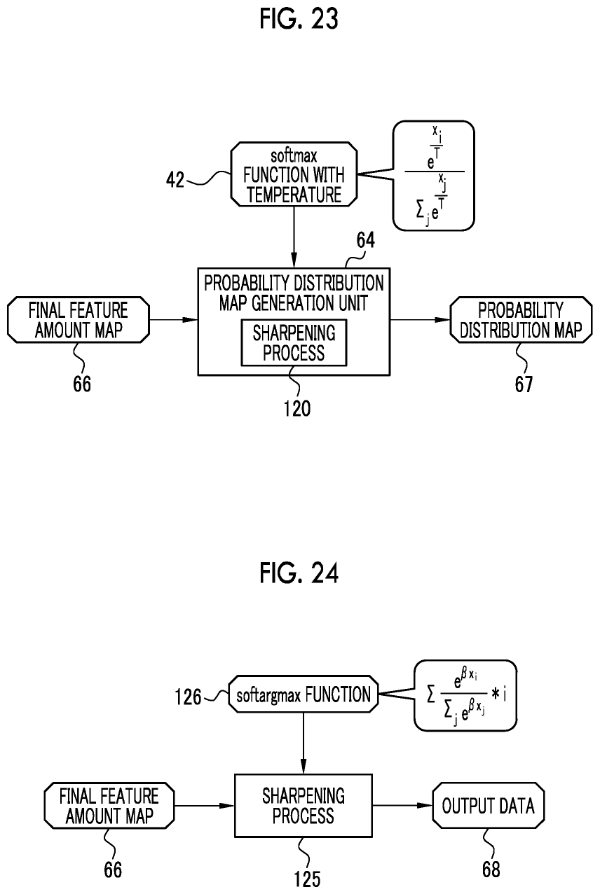

In the first embodiment, the process of applying the softmax function 42 with temperature to the probability distribution map 67 is given as an example of the sharpening process 75. However, the technology of the present disclosure is not limited thereto. For example, a sharpening process 120 illustrated in FIG. 23 may be performed.

In FIG. 23, the sharpening process 120 according to this embodiment is performed by the probability distribution map generation unit 64 of the region extraction model 41. The probability distribution map generation unit 64 performs the sharpening process 120 on the final feature amount map 66 to convert the final feature amount map 66 into the probability distribution map 67. The sharpening process 120 is a process of applying the softmax function 42 with temperature to the final feature amount map 66 instead of the probability distribution map 67.

In this case, similarly to the processed probability distribution map 67P according to the first embodiment, the probability distribution map 67 is, for example, data in which the probabilities that the element 70 of the probability distribution map 67 will be each class have both extreme values such as two values of 0.99999 and 0.00000. Further, in this embodiment, the probability distribution map 67 is an example of the “result of the sharpening process” according to the technology of the present disclosure.

### Fifth Embodiment

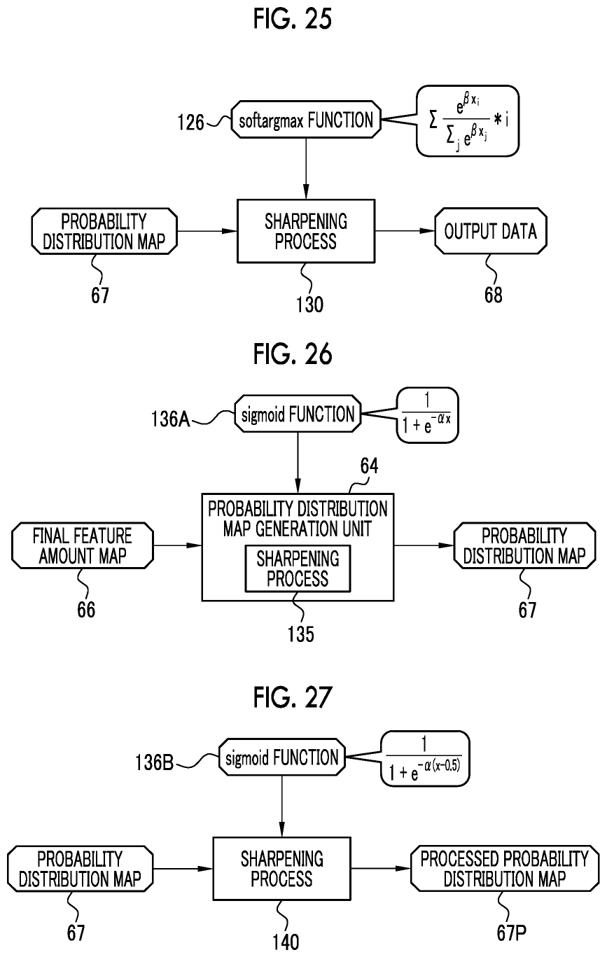

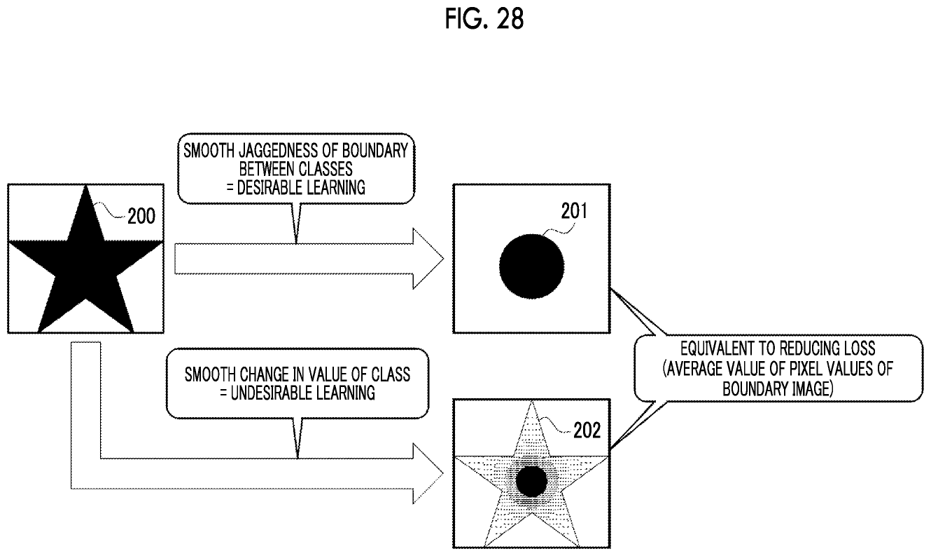

The sharpening process is not limited to the process of applying the softmax function 42 with temperature. A sharpening process 125 illustrated in FIG. 24 as an example or a sharpening process 130 illustrated in FIG. 25 as an example may be performed.

In FIG. 24, the sharpening process 125 is a process of applying a softargmax function 126 to the final feature amount map 66 to convert the final feature amount map 66 into the output data 68. The softargmax function 126 is a function that is illustrated in a balloon of FIG. 24 and is represented by the following Expression (2). In addition, x is an element value related to the probability of being each class in the final feature amount map 66. Further, β is a coefficient and is set to a sufficiently large value. Furthermore, i and j indicate class numbers.

$$\begin{matrix}[ Expression 2 ] & \\ \sum \frac{{e}^{{βx}_{i}}}{{\sum }_{j} {e}^{{βx}_{j}}} * i & ( 2 )\end{matrix}$$

A main portion of the softargmax function 126 is the same as that in the softmax function 42 with temperature except that β replaces 1/T. That is, the softargmax function 126 is the sum of the products of the outputs of each class of the softmax function 42 with temperature and the number i indicating each class. For example, for a certain element of the final feature amount map 66, in a case in which the output of class 2 among four classes 1 to 4 is 0.99999 and the outputs of the other classes 1, 3, and 4 are 0.00000, the solution of the softargmax function 126 is 0.00000×1+0.99999×2+0.00000×3+0.00000×4=1.99998≈2. That is, the solution of the softargmax function 126 is substantially the same as the number indicating the class. Therefore, the use of the softargmax function 126 makes it possible to give the label LB in the sharpening process 125.

The sharpening process 130 illustrated in FIG. 25 is a process of applying the softargmax function 126 to the probability distribution map 67 instead of the final feature amount map 66. In this case, in the softargmax function 126, x is the probability of being each class in the probability distribution map 67. Further, in this embodiment, the output data 68 is an example of the “result of the sharpening process” according to the technology of the present disclosure.

### Sixth Embodiment

In addition, in a case in which there are two classes, a sharpening process 135 illustrated in FIG. 26 as an example or a sharpening process 140 illustrated in FIG. 27 as an example may be performed.

In FIG. 26, the sharpening process 135 is performed by the probability distribution map generation unit 64 of the region extraction model 41, similarly to the sharpening process 120 according to the fourth embodiment. The probability distribution map generation unit 64 performs the sharpening process 135 on the final feature amount map 66 to convert the final feature amount map 66 into the probability distribution map 67. The sharpening process 135 is a process of applying a sigmoid function 136A to the final feature amount map 66. The sigmoid function 136A is a function that is illustrated in a balloon of FIG. 26 and is represented by the following Expression (3). In addition, x is an element value related to the probability of being each class in the final feature amount map 66. Further, a is a gain and is set to a sufficiently large value that is equal to or greater than 1 (α≥1).

$$\begin{matrix}[ Expression 3 ] & \\ \frac{1}{1 + {e}^{- ax}} & ( 3 )\end{matrix}$$

In this case, similarly to the processed probability distribution map 67P according to the first embodiment, the probability distribution map 67 is, for example, data in which the probabilities that the element 70 of the probability distribution map 67 will be each class have both extreme values such as two values of 0.99999 and 0.00000. Further, in FIG. 26, as in the fourth embodiment, the probability distribution map 67 is an example of the “result of the sharpening process” according to the technology of the present disclosure.

The sharpening process 140 illustrated in FIG. 27 is a process of applying a sigmoid function 136B to the probability distribution map 67, instead of the final feature amount map 66, to convert the probability distribution map 67 into the processed probability distribution map 67P. The sigmoid function 136B is a function that is illustrated in a balloon of FIG. 27 and is represented by the following Expression (4). In addition, x is the probability that the element will be each class in the probability distribution map 67. Further, a is a gain and is set to a sufficiently large value that is equal to or greater than 1 (α≥1). In FIG. 27, as in the first embodiment, the processed probability distribution map 67P is an example of the “result of the sharpening process” according to the technology of the present disclosure.

$$\begin{matrix}[ Expression 4 ] & \\ \frac{1}{1 + {e}^{- a ( x - 0.5 )}} & ( 4 )\end{matrix}$$

As described above, the sharpening process may be the process 135 or 140 of applying the sigmoid function 136A or 136B having a gain a of 1 or more to the final feature amount map 66 or the probability distribution map 67.

The head, body, and tail of the pancreas may be extracted as the regions of the classes from a tomographic image including the pancreas. In addition, for example, an automobile, a motorcycle, a bicycle, and a pedestrian may be extracted as the regions of the classes from an image captured by a surveillance camera on the street. As can be seen from these examples, the image is not limited to the medical image, and the classes may not include the lung field.

The hardware configuration of the computer constituting the support server 10 can be modified in various ways. For example, the support server 10 may be configured by a plurality of computers separated as hardware in order to improve processing capacity and reliability. For example, the function of performing the sharpening process 75 and the boundary detection process 80 and the function of performing the average value calculation process 85, the loss calculation process 90, and the update setting process 96 in the learning unit 51 may be distributed to two computers. In this case, the support server 10 is configured by two computers.

As described above, the hardware configuration of the computer of the support server 10 can be appropriately changed according to required performances, such as processing capacity, safety, and reliability. Further, not only the hardware but also an application program, such as the operation program 40, may be duplicated or may be dispersively stored in a plurality of storages in order to ensure safety and reliability.

In each of the above-described embodiments, for example, the following various processors can be used as a hardware structure of processing units performing various processes, such as the RW control unit 50 and the learning unit 51. The various processors include, for example, the CPU 32 which is a general-purpose processor executing software (operation program 40) to function as various processing units, a programmable logic device (PLD), such as a field programmable gate array (FPGA), which is a processor whose circuit configuration can be changed after manufacture, and/or a dedicated electric circuit, such as an application specific integrated circuit (ASIC), which is a processor having a dedicated circuit configuration designed to perform a specific process.

One processing unit may be configured by one of the various processors or by a combination of two or more processors of the same type or different types (for example, a combination of a plurality of FPGAs and/or a combination of a CPU and an FPGA). In addition, a plurality of processing units may be configured by one processor.

A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As described above, various processing units are configured by using one or more of the various processors as a hardware structure.

In addition, specifically, an electric circuit (circuitry) obtained by combining circuit elements, such as semiconductor elements, can be used as the hardware structure of the various processors.

In the technology of the present disclosure, the above-described various embodiments and/or various modification examples may be combined with each other as appropriate. In addition, the present disclosure is not limited to each of the above-described embodiments, and various configurations can be used without departing from the gist of the present disclosure. Furthermore, the technology of the present disclosure extends to a storage medium that non-temporarily stores a program, in addition to the program.

The above descriptions and illustrations are detailed descriptions of portions related to the technology of the present disclosure and are merely examples of the technology of the present disclosure. For example, the above description of the configurations, functions, operations, and effects is the description of examples of the configurations, functions, operations, and effects of portions related to the technology of the present disclosure. Therefore, unnecessary portions may be deleted or new elements may be added or replaced in the above descriptions and illustrations without departing from the gist of the technology of the present disclosure. In addition, in the above descriptions and illustrations, the description of, for example, common technical knowledge that does not need to be particularly described to enable the implementation of the technology of the present disclosure is omitted in order to avoid confusion and facilitate the understanding of portions related to the technology of the present disclosure.

In the specification, “A and/or B” is synonymous with “at least one of A or B”. That is, “A and/or B” means only A, only B, or a combination of A and B. Further, in the specification, the same concept as “A and/or B” is applied to a case in which the connection of three or more matters is expressed by “and/or”.

All of the documents, the patent applications, and the technical standards described in the specification are incorporated by reference herein to the same extent as each document, each patent application, and each technical standard are specifically and individually stated to be incorporated by reference.

What is claimed is:

1. A region extraction model creation support apparatus that supports creation of a region extraction model for extracting regions of a plurality of classes which are in a subject to be recognized in an image and whose boundaries are in contact with each other, the region extraction model creation support apparatus comprising:
a processor; and
a memory that is connected to or provided in the processor,
wherein the processor is configured to:
use, as training data, a learning input image and local annotation data generated by locally giving labels to the regions of the classes in the learning input image;
direct the region extraction model to output a final feature amount map having element values related to probabilities of being the regions of the classes;
perform a sharpening process on the final feature amount map or a probability distribution map that has been generated on the basis of the final feature amount map and that shows the probability for each class;
detect the boundary on the basis of a result of the sharpening process; and
update the region extraction model in a direction in which a boundary length loss corresponding to a length of the boundary is reduced.

2. The region extraction model creation support apparatus according to claim 1,
wherein the processor is configured to:
direct the region extraction model to output learning output data obtained by extracting the regions of the classes in the learning input image;
calculate a loss of the region extraction model according to a result of comparison between the local annotation data and the learning output data for local parts to which the labels have been given;
add up the loss and the boundary length loss to obtain a first total loss; and
update the region extraction model in a direction in which the first total loss is reduced.

3. The region extraction model creation support apparatus according to claim 2,
wherein the processor is configured to:
further add a size loss corresponding to sizes of the regions of the plurality of classes to the first total loss to obtain a second total loss; and
update the region extraction model in a direction in which the second total loss is reduced.

4. The region extraction model creation support apparatus according to claim 1,
wherein the sharpening process is a process of applying a softmax function with temperature having a temperature parameter equal to or less than 1 to the final feature amount map or the probability distribution map.

5. The region extraction model creation support apparatus according to claim 1,
wherein the sharpening process is a process of applying a softargmax function to the final feature amount map or the probability distribution map.

6. The region extraction model creation support apparatus according to claim 1,
wherein the sharpening process is a process of applying a sigmoid function having a gain equal to or greater than 1 to the final feature amount map or the probability distribution map.

7. The region extraction model creation support apparatus according to claim 1,
wherein the boundary length loss is an average value of pixel values of a boundary image generated by detecting the boundary from the result of the sharpening process.

8. The region extraction model creation support apparatus according to claim 1,
wherein the processor is configured to:
receive designation of a region from which the boundary is to be detected in the result of the sharpening process.

9. The region extraction model creation support apparatus according to claim 1,
wherein the image is a medical image.

10. The region extraction model creation support apparatus according to claim 9,
wherein the classes include a lung lobe.

11. A method for operating a region extraction model creation support apparatus that supports creation of a region extraction model for extracting regions of a plurality of classes which are in a subject to be recognized in an image and whose boundaries are in contact with each other, the method comprising:
using, as training data, a learning input image and local annotation data generated by locally giving labels to the regions of the classes in the learning input image;
directing the region extraction model to output a final feature amount map having element values related to probabilities of being the regions of the classes;
performing a sharpening process on the final feature amount map or a probability distribution map that has been generated on the basis of the final feature amount map and that shows the probability for each class;
detecting the boundary on the basis of a result of the sharpening process; and
updating the region extraction model in a direction in which a boundary length loss corresponding to a length of the boundary is reduced.

12. A non-transitory computer-readable storage medium storing a program for operating a region extraction model creation support apparatus that supports creation of a region extraction model for extracting regions of a plurality of classes which are in a subject to be recognized in an image and whose boundaries are in contact with each other, the program causes a computer to execute a process comprising:
using, as training data, a learning input image and local annotation data generated by locally giving labels to the regions of the classes in the learning input image;
directing the region extraction model to output a final feature amount map having element values related to probabilities of being the regions of the classes;
performing a sharpening process on the final feature amount map or a probability distribution map that has been generated on the basis of the final feature amount map and that shows the probability for each class;
detecting the boundary on the basis of a result of the sharpening process; and
updating the region extraction model in a direction in which a boundary length loss corresponding to a length of the boundary is reduced.
